(19) United States
(12) Patent Application Publication
(10) Pub. No.: US 2013/0295630 A1
(43) Pub. Date: Nov. 7, 2013

(54) METHOD OF PRODUCING ETHANOL

(71) Applicant: DEAN SNYDER CONSTRUCTION, Clear Lake, IA (US)

(72) Inventor: Eric A. Nobbs, Nora Springs, IA (US)

(73) Assignee: DEAN SNYDER CONSTRUCTION, Clear Lake, IA (US)

(21) Appl. No.: 13/874,543

(22) Filed: May 1, 2013

Related U.S. Application Data
(60) Provisional application No. 61/640,873, filed on May 1, 2012.

(51) Int. Cl. C12P 7/06

(52) U.S. Cl. CPC C12P 7/06
USPC 435/161

(57) ABSTRACT

A method of producing ethanol wherein feedstock is broken down and mixed with yeast in a fermenting tank at an optimum predetermined temperature. An alcohol additive is added to the mixture in the fermentation tank. Once fermented, the mixture is distilled and stored.

### CROSS REFERENCE TO RELATED APPLICATION

This application claims the benefit of U.S. Provisional Application No. 61/640,873 filed May 1, 2012.

### BACKGROUND OF THE INVENTION

This invention relates to ethanol production. More specifically this invention relates to increasing the yield of ethanol production.

As the cost of gasoline and fossil fuels continue to rise there has been an increased interest in ethanol production as an alternative fuel source. In particular, in an ethanol production plant or facility grain such as corn is provided and is typically placed into a hammer mill, cooked and liquefied before undergoing a fermentation process. Typically that ethanol is then distilled and stored for use as ethanol fuel. In the fermentation process a yeast known as saccharomyces cerevisiae or s. cerevisiae is utilized and reacts with simple sugars in the grain to create an ethanol yield.

While the above process is effective at producing ethanol problems still remain. In particular, the yields or amount of ethanol alcohol that is able to be produced from grain is typically a very low yield. As a result of the cost of harvesting grain and utilizing the ethanol production process these low yield amounts result in high cost thus reducing potential profits from the manufacture and production of the ethanol. In addition the fermentation process can take long periods of time thus again providing a limit on the amount of ethanol that is produced over a predetermined amount of time.

Thus a principal object of the present invention is to increase the efficiency of ethanol yield in an ethanol production process.

Yet another object of the present invention is to increase the speed of ethanol from an ethanol production process.

### SUMMARY OF THE INVENTION

A method of producing ethanol wherein feedstock is broken down and mixed with yeast in a fermenting tank at an optimum predetermined temperature. An alcohol additive is added to the mixture in the fermentation tank. Once fermented, the mixture is distilled and stored.

### BRIEF DESCRIPTION OF THE FIGURES

FIG. 1 is a schematic view of an environment for a method of producing ethanol; and

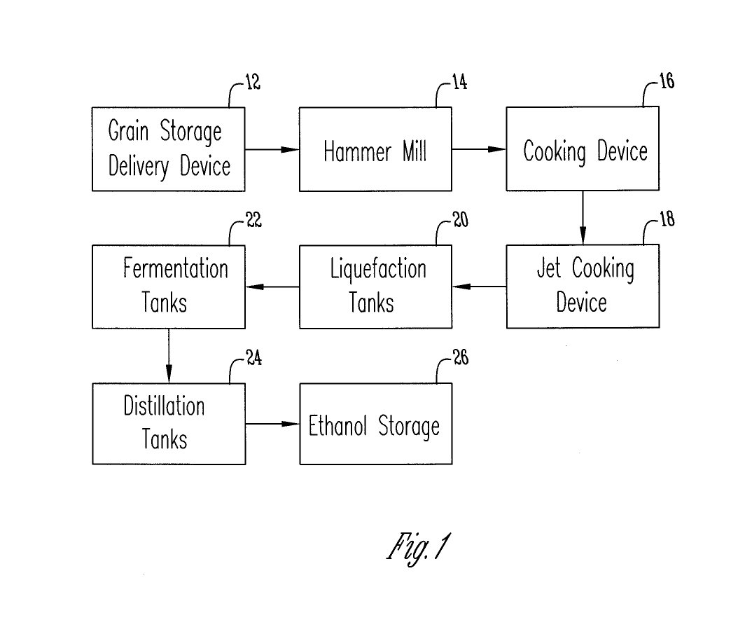

FIG. 2 is a flow diagram of a method of producing ethanol.

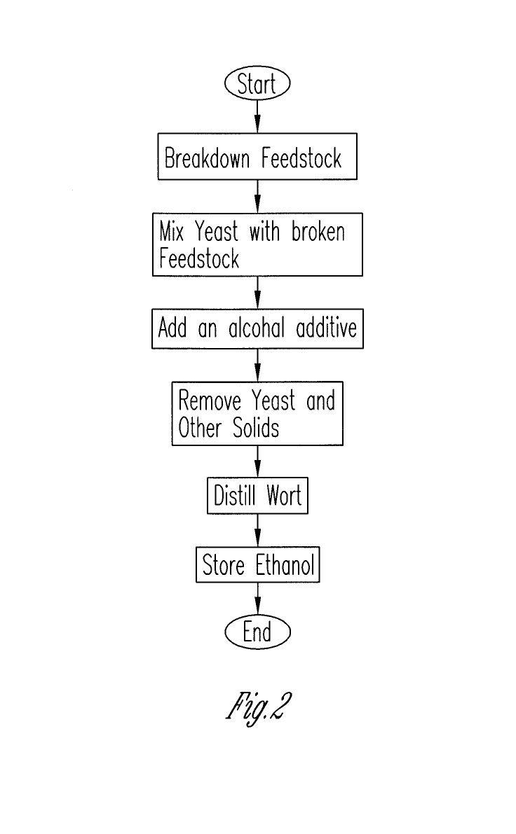

### DESCRIPTION OF THE PREFERRED EMBODIMENT

The figures show a system 10 utilized to produce ethanol. The system 10 includes a grain or cellulose delivery device 12 that can present grain to a hammer mill device 14. A cooking device 16 and a jet cooker 18 are then utilized for providing a liquefied grain to liquification tanks 20 for delivery to fermentation tanks 22 or containers. A fermenting agent yeast is added to the fermentation containers 22 to cause the fermentation process and produce ethanol over an extended period of time. Once fermented, the mixture is distilled in distillation tanks 24 and then stored in ethanol tanks 24. Such a system is known in the art and the use of the hammer mill, tanks, cookers, or the like may vary depending on individualized processes.

In operation, feedstock, which can include grain, cellulose or the like is broken down as required to make simple sugars capable of being digested by yeast. This step can involve physically grinding or breaking the feedstock such as in the hammer mill device 12 to increase surface area as well as cooking and/or addition of enzymes in the cooking device 16. Both processes break down chemical bonds in complex sugars to produce simple sugars.

Yeast is then added to and mixed through the feedstock in the fermentation tank 22 and the temperature is adjusted/maintained at a predetermined optimum temperature for fermentation. The optimum temperature varies, depending upon the type of yeast being used and the level of alcohol and other characteristics desired. In a preferred embodiment the predetermined temperature is between 90-100 degrees Fahrenheit.

At this time within the fermentation tank 22 an alcohol additive such as phenylethyl alcohol (PEA) (C8 H10 O) or Tryptophol (C10 H11 N O)is added. In a preferred embodiment the PEA or Tryptophol is added at about 1% per volume of the total solution. As a result of the alcohol additive during the fermentation process the microbes react to an auto-inducer PEA to go into a hyper reproductive mode in order to exploit food sources within the grain to produce ethanol. In particular, instead of merely having the yeast eat simple sugars and ignore complex sugars, carbohydrates and proteins when the alcohol (PEA or trytophol) is added to the mixture the complex sugars, carbohydrates and proteins are broken down to create additional ethanol. In particular, typically yeast population builds up slowly and as ethanol levels increase they begin to die off. However, when utilizing the alcohol additive the population reaches higher than normal levels before the ethanol reaches lethal levels for the yeast thus resulting in more yeast cells producing ethanol before drying.

In particular the desired effect of the PEA is to act on the yeast. The yeast exposed to the PEA reproduces more quickly and takes advantage of alternate food sources, allowing it to: 1. speed up the fermentation process, 2. digest more of the feedstock than just the simple sugars, 3. yield a higher alcohol content. This also reduces the amount of time needed to heat each batch, allows for more batches in the same time period resulting in increased production and may also reduce the number of enzymatic additives required in step 1.

Once fermentation is complete, the yeast is dead and the sugars are all digested, unless it is desirable to leave some sugars for flavor (in beverages) in which case the process is stopped early. The yeast and other solids are removed from the resulting liquid (aka “wort”, “beer”, or “must”) and either used to produce by-products, re-used in the process, or disposed of. Next the wort is distilled to extract nearly pure (approximately 95%) ethanol. This step also requires the addition of gasoline to “denature” ethanol.

In a preferred embodiment the fermentation process is conducted with s. cerevisiea as the yeast or fermenting agent and the phenylethyl alcohol is added to the mixture in an amount of between 0.25% to 8% by volume of the chemical mixture being fermented. In a preferred embodiment the amount of PEA added is 1% volume of the mixture. As a result of testing of such a process it has been shown that up to a 300% increase in yield of ethanol can occur. Specifically, tested has been concentrations 0.25% by volume to 1.5 by volume PEA and all volumes have increased yield of ethanol with the 1% concentration by volume appearing to be the optimal amount of additive to be added to the mixture.

Thus provided is a method of enhancing the speed and efficiency of ethanol production. In particular, by utilizing an alcohol such as phenylethyl alcohol or trytophol, not only is the speed of the fermentation process of fermenting grain into alcohol increased but additionally the yield of ethanol from the grain is greatly increased. Thus, at the very least all of the stated objectives have been met.

What is claimed:

1. A method of producing ethanol comprising the steps of:
breaking down feedstock to make simple sugars capable of being digested by yeast;
mixing yeast with the broken down feedstock in a fermentation tank at a predetermined optimum temperature;
adding an alcohol additive to the mixture in the fermentation tank; and
removing yeast and other solids from the fermentation tank.

2. The method of claim 1 wherein the feedstock is broken down by grinding the feedstock in a hammer mill.

3. The method of claim 1 wherein the feedstock is broken down by cooking the feedstock in a cooking device.

4. The method of claim 1 wherein the predetermined optimum temperature is based upon the type of yeast used and a level of desired alcohol.

5. The method of claim 1 wherein the predetermined optimum temperature is between 90 to 100 degrees Fahrenheit.

6. The method of claim 1 wherein the alcohol additive is phenylethyl alcohol.

7. The method of claim 1 wherein the alcohol additive is tryptophol.

8. The method of claim 1 wherein the amount of alcohol additive added to the mixture is between 0.25% to 8% by volume of the mixture being fermented.

9. The method of claim 1 wherein the amount of alcohol additive added to the mixture is about 1.0% by volume of the mixture being fermented.

10. The method of claim 1 wherein the yeast is S. cerevisiae.

11. The method of claim 1 comprising the further step of distilling the fermented mixture to form ethanol.
